(19) United States
(12) Patent Application Publication
(10) Pub. No.: US 2014/0309587 A1
(43) Pub. Date: Oct. 16, 2014

(54) TUBE CONTINUUM ROBOT AND METHOD FOR MANUFACTURING TUBE HAVING ANISOTROPIC PATTERNS

(71) Applicant: Korea Institute of Science and Technology, Seoul (KR)

(72) Inventors: Keri KIM, Seoul (KR); Sung Chul KANG, Seoul (KR); Kyu-Jin CHO, Seoul (KR); Dae-Young LEE, Ansan-si (KR); Ji-Suk KIM, Seoul (KR); Yong-Jai PARK, Goyang-si (KR)

(73) Assignee: KOREA INSTITUTE OF SCIENCE AND TECHNOLOGY, Seoul (KR)

(21) Appl. No.: 13/930,124

(22) Filed: Jun. 28, 2013

(30) Foreign Application Priority Data
Apr. 16, 2013 (KR) 10-2013-0041767

(51) Int. Cl. A61M 25/01; B23K 26/38; B23K 26/36; A61B 19/00; A61B 17/34

(52) U.S. Cl. CPC A61M 25/0105; A61B 19/2203; A61B 17/3417; B23K 26/365; B23K 26/38
USPC 604/95.01; 219/121.69; 219/121.72

(57) ABSTRACT

Disclosed herein are a tube continuum robot and a method for manufacturing a tube. More particularly, disclosed are a tube continuum robot and a method for manufacturing a tube, which is used in the tube continuum robot having a plurality of overlapping tubes and has anisotropic patterns for controlling the bending rigidity and torsional rigidity of the tube. In an embodiment, a tube continuum robot has a plurality of overlapping tubes, one or more of the plurality of overlapping tubes having a curved shape, wherein a plurality of anisotropic patterns are formed on the outer circumferential surface of the one or more tubes along the lengthwise or circumferential direction of the tubes.

### CROSS-REFERENCE TO RELATED APPLICATION

This application claims priority to Korean Patent Application No. 10-2013-0041767, filed on Apr. 16, 2013, and all the benefits accruing therefrom under 35 U.S.C. §119, the contents of which in its entirety are herein incorporated by reference.

### BACKGROUND

1. Field

The present disclosure relates to a tube continuum robot and a method for manufacturing a tube, and more particularly to a tube continuum robot and a method for manufacturing a tube, which is used in the tube continuum robot having a plurality of overlapping tubes and has anisotropic patterns for controlling the bending rigidity and torsional rigidity of the tube.

2. Description of the Related Art

Minimally invasive surgery refers to a procedure performed through minimal incisions without opening the abdomen and is advantageous in that little or no scars or sequelae remain because the incisions are small, and recovery is fast. Microsurgical instruments should be used to perform such minimally invasive surgery, and many studies on the manufacture and control of such microsurgical instruments have been conducted.

FIG. 1 shows a conventional tube continuum robot disclosed in US Patent Publication No. 2013/0018303.

Referring to FIG. 1, the tube continuum robot is curved and made of a superelastic shape memory alloy. In the tube continuum robot, tubes having different diameters and curvatures overlap with each other, and the position of an end effector 125 changes depending on the input angle due to the interaction between the tubes. When three tubes 110, 115 and 120 are used, these tubes have a diameter of 2-5 mm and a length of 10-20 cm. Such elongated tubes 110, 115 and 120 enter through the human nostril or mouse and can serve as a passage through which instruments which are used in minimally invasive surgery can enter. In addition, using an energy equation, an angle at which minimizes the energy that can be possessed by the overlapping tubes 110, 115 and 120, and the final position of the end effector 125, are estimated.

The three tubes 110, 115 and 120 are divided into an outer flexible tube 110, a middle flexible 115 and an inner flexible tube 120. These tubes 110, 115 and 120 have inner rotational degrees of freedoms 305, 315 and 325 and inner translation degrees of freedoms 310, 320 and 330, respectively.

The biggest problem of this robot that two local energy minimum points are present in a certain input angle difference when analyzing the energy equation. The two energy minimum points are spaced apart from each other, and thus, when an input equal to or greater than the corresponding input angle is applied, the position of the tubes instantaneously sparks. This is called the bifurcation phenomenon. The bifurcation phenomenon appears because stored distortion energy is instantaneously released when two curved superelastic tubes have different input values. For example, the inner flexible tube 120 does not slowly rotate in the rotational direction indicated by reference numeral 325 in FIG. 1, but instantaneously rotates abruptly from a first point to a second point.

This bifurcation problem can make the control of the robot unstable and is highly risk in a surgical operation.

### SUMMARY

In order to solve the above-described bifurcation problem occurring in the prior art, the physical property values of tubes need to be changed. It is expected that this need can be achieved by minimizing the EI/GJ ratio, in which EI is a value for bending rigidity, and GJ is a value for torsional rigidity. Herein, E is Young's modulus, I is the area moment of inertia, G is shear modulus, and J is the polar moment of inertia.

It is an object of the present disclosure to provide a tube continuum robot and a method for manufacturing a tube, in which the physical property values (such as bending rigidity and torsional rigidity) of the tube can be changed by forming anisotropic patterns on the outer circumferential surface of the tube.

To achieve the above object, an embodiment of the present disclosure provides a tube continuum robot having a plurality of overlapping tubes, one or more of the overlapping tubes having a curved shape, wherein a plurality of anisotropic patterns are formed on the outer circumferential surface of the one or more tubes along the lengthwise or circumferential direction of the tubes.

The anisotropic patterns may be formed by performing a cutting, peeling, etching, deposition or annealing process on the outer circumferential surface of the tubes.

The ratio of the area or length of each of the anisotropic patterns along the lengthwise direction of the tubes to the area or length of the anisotropic pattern along the circumferential direction of the tubes may be controlled so that a ratio at which the bending rigidity and torsional rigidity of the tubes are decreased is controlled.

Each of the anisotropic patterns on the tubes may be configured such that the area or length of the anisotropic pattern along the lengthwise direction of the tubes is smaller than the area or length of the anisotropic pattern along the circumferential direction of the tubes so that the bending rigidity of the tubes decreases more than the torsional rigidity of the tubes.

Each of the anisotropic patterns on the tubes may be configured such that the area or length of the anisotropic pattern along the lengthwise direction of the tubes is larger than the area or length of the anisotropic pattern along the circumferential direction of the tubes so that the torsional rigidity of the tubes decreases more than the bending rigidity of the tubes.

The anisotropic patterns may be formed to be inclined at a predetermined angle with respect to the lengthwise direction or circumferential direction of the tubes.

Both ends of the anisotropic patterns may be bent or have a circular shape.

A method for manufacturing a tube according to an embodiment of the present disclosure is a method for a tube which is used in a tube continuum robot having a plurality of overlapping tubes, the method including: determining the size, distance and type of patterns to be formed on the tube, and selecting a region required to be patterned; and forming a plurality of anisotropic patterns on the outer circumferential surface of the tube along the lengthwise or circumferential direction of the tube.

Forming the plurality of anisotropic patterns may comprise performing a cutting, peeling, etching, deposition or annealing process on the circumferential surface of the tube.

When the cutting or peeling process is performed using a laser, the cutting or peeling process may be programmed so that it is continuously performed by recognizing a pattern region deviating from the scanning range of the laser scanner, expressing the recognized region as a new processing range, and selecting the end point of the previous range as the start point of the new range.

Forming the plurality of anisotropic patterns may be performed so that the ratio of the area or length of each of the anisotropic patterns along the lengthwise direction of the tubes to the area or length of the anisotropic pattern along the circumferential direction of the tubes may be controlled so that a ratio at which the bending rigidity and torsional rigidity of the tubes are decreased is controlled.

Forming the plurality of anisotropic patterns may comprise forming anisotropic patterns on a tube curved with a predetermined curvature, in which the sizes, lengths or shapes of the anisotropic patterns inside, outside and near the curved portion may differ from each other.

Forming the plurality of anisotropic patterns may comprise, before forming patterns on a tube curved with a predetermined curvature, straightening the curved tube by inserting a circular rod therein.

Forming the plurality of anisotropic patterns may comprise forming anisotropic patterns on a straight tube by a cutting, peeling, etching, deposition or annealing process, curving the tube having the anisotropic patterns with a predetermined curvature, and annealing the curved tube.

### BRIEF DESCRIPTION OF THE DRAWINGS

FIG. 1 shows a tube continuum robot according to the prior art.

FIG. 2 is a perspective view showing a tube having anisotropic patterns according to an embodiment of the present disclosure.

FIG. 3 is a perspective view showing a tube having anisotropic patterns according to another embodiment of the present disclosure.

FIG. 4 is a perspective view showing a tube having anisotropic patterns according to still another embodiment of the present disclosure.

FIG. 5 shows tubes having anisotropic patterns according to different embodiments of the present disclosure.

FIG. 6 is a perspective view showing a processing machine for manufacturing a tube having anisotropic patterns according to the present disclosure.

FIGS. 7A and 7B illustrate laser processing.

FIGS. 8A and 8B show test devices for measuring the bending strength and torsional strength of a tube.

FIG. 9 is a perspective view showing a tube, the bending strength and torsional strength of which are to be measured.

FIG. 10 is a flow chart showing a method for manufacturing a tube having anisotropic patterns according to an embodiment of the present disclosure.

### DETAILED DESCRIPTION

Hereinafter, a tube continuum robot and a method for manufacturing a tube having anisotropic patterns according to preferred embodiments of the present disclosure will be described in detail.

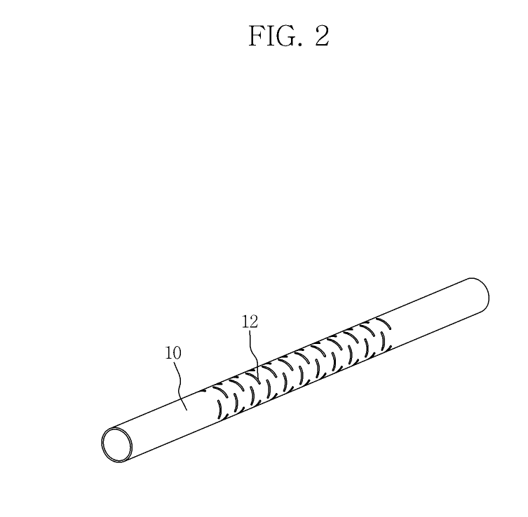

FIG. 2 is a perspective view showing a tube having anisotropic patterns according to an embodiment of the present disclosure.

Referring to FIG. 2, a plurality of anisotropic patterns 12 are formed on the outer circumferential surface of a tube 10 by cutting. When such anisotropic patterns 12 are formed, the bending rigidity and torsional rigidity of the tube 10 can dramatically decrease. In addition, when the plurality of anisotropic patterns 12 are formed along the circumferential direction of the tube 10 as shown in FIG. 2, the bending rigidity will decrease much more than the torsional rigidity. In other embodiments of the present disclosure, the plurality of anisotropic patterns 12 as shown in FIG. 2 may be formed by etching or deposition in place of cutting.

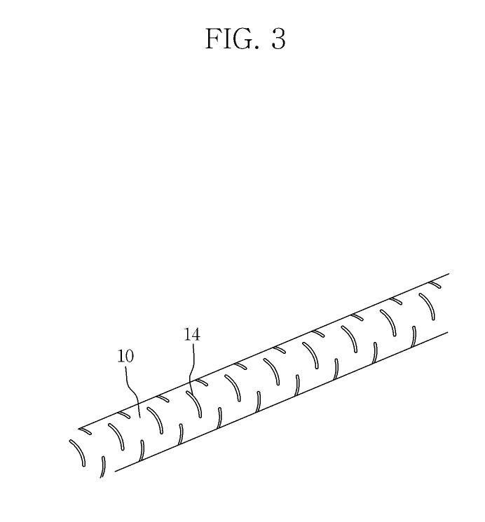

FIG. 3 is a perspective view showing a tube having anisotropic patterns according to another embodiment of the present disclosure.

Referring to FIG. 3, the outer circumferential surface of the tube 10 is removed by a predetermined thickness by a peeling process while a plurality of anisotropic patterns 14 are formed thereon. In this peeling process, the bending and the torsional rigidity all decrease, like the case of the cutting process, but the ratio therebetween is relatively low. In FIG. 3, the anisotropic patterns 14 are formed along the circumferential direction, and thus the bending rigidity will decrease more than the torsional rigidity. Meanwhile, when anisotropic patterns are formed along the lengthwise direction of the tube 10, the torsional rigidity will decrease more than the bending rigidity. In addition, the anisotropic patterns 14 as shown in FIG. 14 may also be formed by etching or deposition.

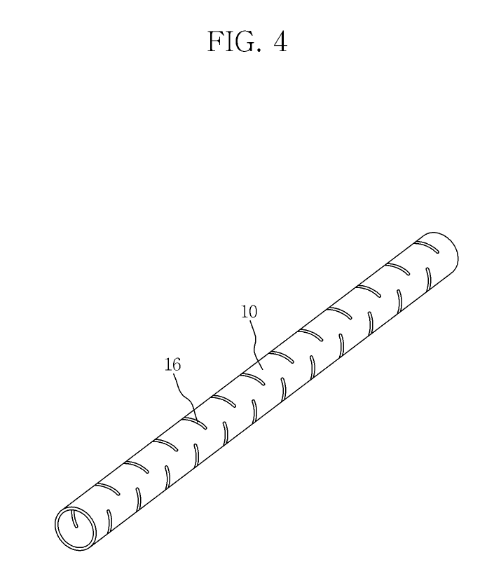

FIG. 4 is a perspective view showing a tube having anisotropic patterns according to still another embodiment of the present disclosure.

Referring to FIG. 4, a plurality of anisotropic patterns 16 are formed on the outer circumferential surface of the tube 10 by local annealing. The local annealing may be performed using a heat pad, like the cutting process or the peeling process. For example, the tube 10 can be locally heated using laser conditions for maintaining a predetermined temperature. This local annealing can show an effect like a tube made of materials having different physical property values, mixed in a certain pattern.

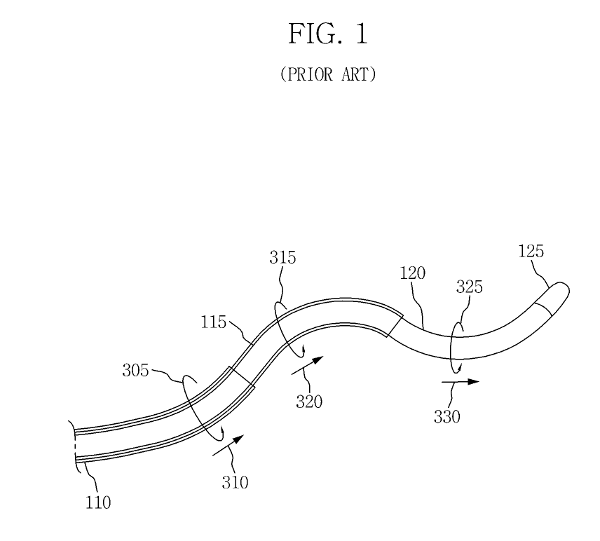

The tubes 10 having anisotropic patterns as shown in FIGS. 2 to 4 may be used in a tube continuum robot having a plurality of overlapping tubes as shown in FIG. 1. The tube 10 may be made of a metal. For example, when a tube continuum robot comprising the tubes 10 made of superelastic nitinol is manufactured, the position of the end effector can be controlled by relative movement between the curved superelastic nitinol tubes.

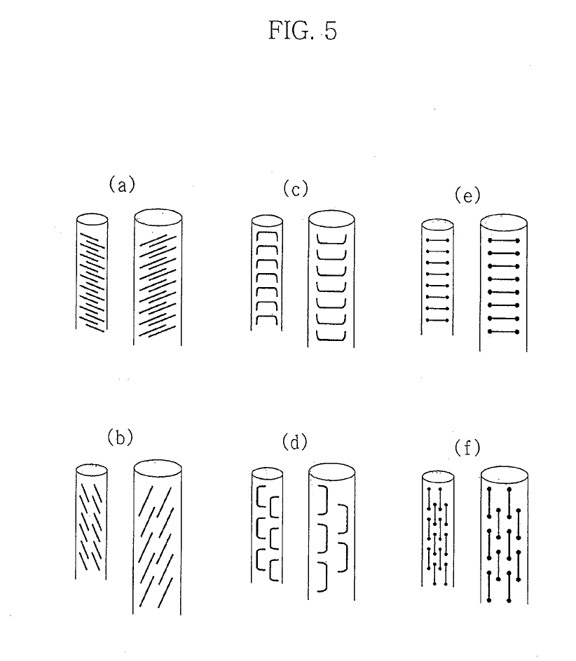

FIG. 5 shows tubes having formed thereon anisotropic patterns according to other embodiments of the present disclosure.

Referring to FIG. 5, a plurality of anisotropic patterns are formed on the outer circumferential direction of the tubes along the lengthwise or circumferential direction. FIG. 5 shows anisotropic patterns formed on the straight tubes, but the technical idea of the present disclosure is not limited thereto, and anisotropic patterns may also be formed on tubes curved with a predetermined curvature.

The above-described anisotropic patterns can be formed by performing a cutting, peeling, etching, deposition or annealing process on the outer circumferential surface of a tube. In the case of the etching, deposition or annealing process, anisotropic patterns may be formed after a patterned mask is wound around a tube.

The cutting process can be performed by cutting the surface of a tube according to a specific pattern by laser processing. According to cutting patterns, the tube may have various physical property values. Laser pattern cutting can cause side effects, including an undesired increase in friction between tubes due to a heat-affected zone (HAZ), locking, cracking and the like, but such side effects can be significantly alleviated by partially annealing the pattern shapes to change the physical property values of the tubes.

The peeling process is a process in which a predetermined pattern on a tube is removed by laser processing. Although the effect of changing the physical property values is insignificant compared to that of laser cutting, the peeling process has an advantage in that no hole is formed.

The etching process is a process in which the outer circumferential surface is masked according to a desired pattern, and then etched. As used herein, the term “etching process” refers to a chemical processing technique in which a desired structure is obtained by etching the unprotected portion of a target material using a strong acid or an etchant. Because ultrafine processing is possible according to masking technology, the etching process is frequently used in MEMS processing or semiconductor processes and may be used to form fine anisotropic patterns on a tube according to the present disclosure.

In the deposition process, the outer circumferential surface of a tube is not cut out, but a thin film is deposited on tube portions other than potions in which anisotropic patterns are to be formed, so that tube portions corresponding to the anisotropic patterns are exposed. The deposition process is a processing method that is used mainly in semiconductor processes, and it refers to a processing technique of obtaining a desired structure by depositing various thin films. The deposition process is largely divided into physical vapor deposition (PVD) and chemical vapor deposition (CVD). Examples of the PVD include a vapor deposition process in which a metal to be deposited is heated to the melting point thereof so that the gaseous metal is deposited on the substrate surface, and a sputtering process in which a material to be deposited is bombarded with ions having energy so as to be deposited on a wafer. The CVD is a method in which particles produced by the reaction of gaseous compounds form a thin film on a target surface. In the present disclosure, any of PVD and CVD may be used depending on the material of a tube.

In the annealing process, the properties (hardness or softness) of a tube are changed by annealing the outer circumferential surface of the tube. Specifically, the desired properties of the tube can be obtained by heating tube portions corresponding to anisotropic patterns to a temperature equal to or higher than a predetermined temperature, maintaining the tube at the heated temperature for a predetermined time, and then cooling the tube. For local annealing, the tube can be heated in a state in which a heat pad is attached to the tube, or the tube can be heated in a state in which the focus of the laser is scattered so that the tube is not cut.

Referring to FIG. 5(a) to FIG. 5(f) again, the left tube having a smaller diameter is inserted into the right tube having a larger diameter so that the tubes overlap each other to form a tube continuum robot. The sizes of the diameters of the left and right tubes are not limited to the ratios shown in FIG. 5, and the ratio of the diameters of the tubes can be changed to various ratios.

FIGS. 5(a) and 5(b) show a configuration in which anisotropic patterns formed on the outer circumferential surface of the tube are inclined at a certain angle with respect to the lengthwise or circumferential direction of the tube. In addition, FIGS. 5(c) and 5(d) show a configuration in which anisotropic patterns are bent at both ends. When both ends of the anisotropic patterns formed on the tube having a smaller diameter and the tube having a larger diameter are bent in opposite directions as shown in FIGS. 5(c) and 5(d), the concentration of stress and the interference between the inner and outer tubes can be reduced. FIGS. 5(e) and 5(f) show a configuration in which both ends of anisotropic patterns have a circular shape, and this configuration makes it possible to control the concentration of stress.

The shapes of the anisotropic patterns shown in FIG. 5 are illustrative only, a combination of these shapes may be used or other various shapes may be used. Generally, patterns formed along the lengthwise direction of a tube weaken the torsional rigidity, and patterns formed along the circumferential direction weaken the bending rigidity. In addition, if the number of patterns excessively increases, the roughness of the tube itself will greatly decrease, resulting in early breakage, and if the size of patterns increases, the interference between the overlapping tubs will become severe. Thus, considering the above-described constraint conditions of pattern formation, the shapes of anisotropic patterns can be designed in various ways.

In an embodiment of the present disclosure, when patterns are formed on a tube curved with a predetermined curvature, the sizes, lengths or shapes of the anisotropic patterns formed inside, outside or near the curved portion may differ from each other in order to control the concentration of stress. In addition, before patterns are formed on a tube curved with a predetermined curvature, the tube may also be straightened by inserting a circular rod therein, followed by patterning.

In another embodiment of the present disclosure, when anisotropic patterns are formed on a straight tube, these patterns may be formed by a cutting, peeling, etching, deposition or annealing process as described above, and the tube having the patterns may be curved with a predetermined curvature and annealed, after which it may be used in the manufacture of a tube continuum robot.

In still another embodiment of the present disclosure, each of the anisotropic patterns on the tube may be configured such that the ratio of the area or length of the anisotropic pattern along the lengthwise direction of the tube to the area or length along the circumferential direction of the tube is controlled so that a ratio at which the bending rigidity and torsional rigidity of the tube are decreased is controlled. For example, each of the anisotropic patterns on the tube may be configured such that the area or length of the anisotropic pattern along the lengthwise direction of the tube is smaller than the area or length of the anisotropic pattern along the circumferential direction of the tube so that the bending rigidity decreases more than the torsional rigidity. In addition, each of the anisotropic patterns on the tube may also be configured such that the area or length of the anisotropic pattern along the lengthwise direction of the tube is larger than the area or length of the anisotropic pattern along the circumferential direction of the tube so that the torsional rigidity decreases more than the bending rigidity.

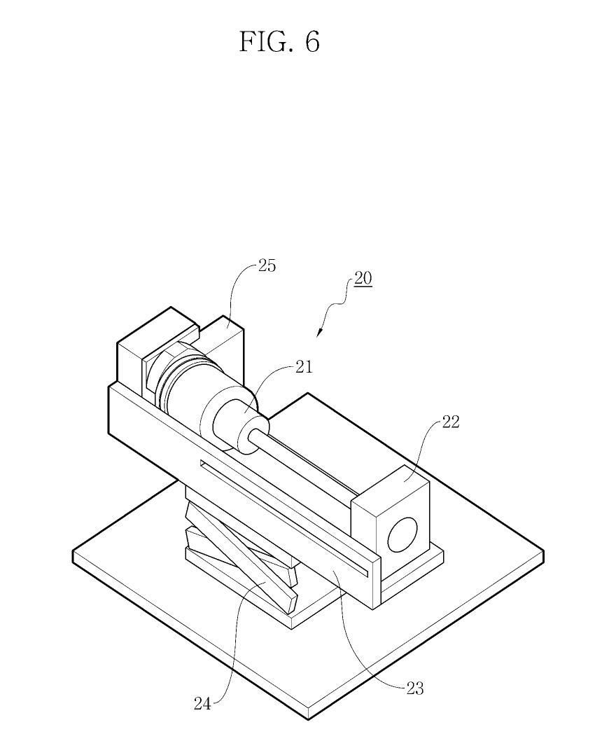

FIG. 6 is a perspective view showing a processing machine for manufacturing a tube having anisotropic patterns according to the present disclosure.

Referring to FIG. 6, a processing machine 20 comprises a drill chuck 21, a bearing support 22, a linear guide 23, a lab jack 23 and a rotating stage 25.

The drill chuck 21 is an electric drill chuck and serves to fix one end of a tube. The tube may be straight or curved in shape and can be drooped by the curvature and self-weight of the tube. For this reason, the other end of the tube is supported by a bearing (not shown) received in the bearing support 22. The linear guide 23 makes it possible to control the position of the bearing, and thus tubes having various lengths can be processed.

The lab jack 24 can move upward and downward, and thus the focusing distance of a lens during laser processing can be controlled.

The rotating stage 25 receives power from a motor (not shown) to rotate the drill chuck 21 connected to the top thereof, and thus the tube 10 whose one end is fixed to the drill chuck 21 can also be rotated. The rotating stage 25 shows a rotation accuracy error that does not exceed the laser width (about 10 μm), and it preferably has an automatic reference point return function in order to minimize the backlash error.

Using the above-described processing machine 20 and a laser, the tube having anisotropic patterns according to the present disclosure can be manufactured.

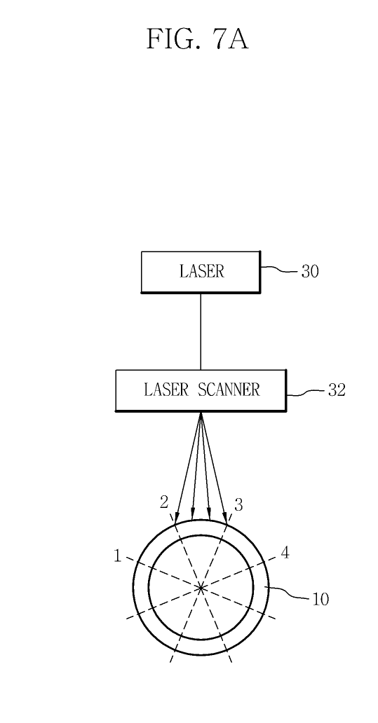

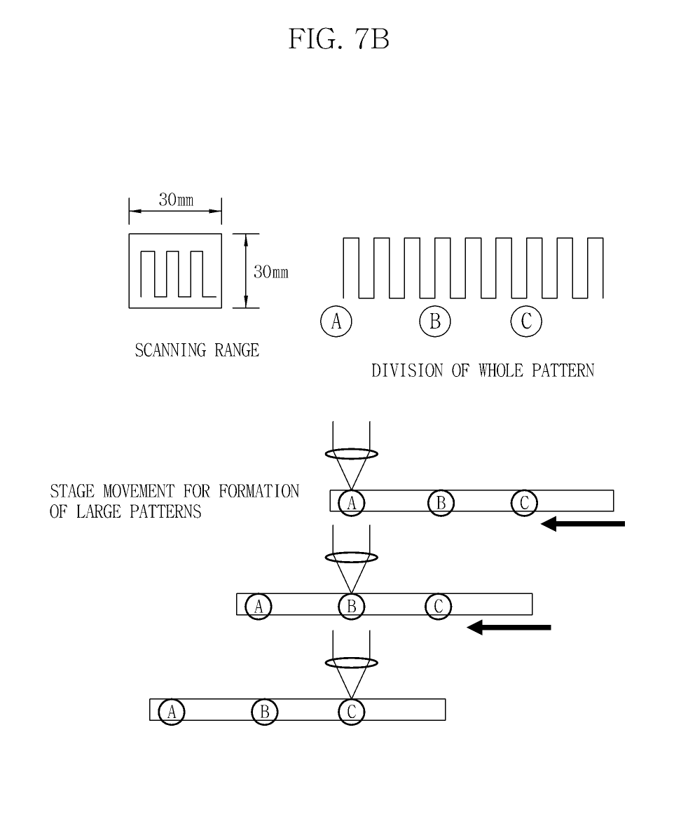

FIGS. 7A and 7B illustrate laser processing.

Referring to FIGS. 7A and 7B, a laser beam outputted from a laser 30 is scanned on the outer circumferential surface of the tube 10 at dense intervals through a laser scanner 32. To process the tube 10 with the laser 30, the laser 30 is fixed, and the tube 10 is moved by a transport stage (not shown) in the axial direction with the respect to the rotating stage 25 while the laser beam output should be effectively controlled. Thus, it is preferred to previously construct a program that engages with the laser 30 using the rotating stage 25 and the motor. The variables to be considered when processing the tube 10 using the laser 30 include material variables, such as the kind, thickness and surface condition of material, and laser beam variables such as beam output, beam mode and beam cycle. Thus, considering such variables and characteristics, optimal processing conditions are determined through experiments, and based on the determined processing conditions, patterns can be formed on the tubes 10, which have various sizes and are made of various materials.

As shown in FIG. 7B, the scanning range of the laser scanner 32 is limited, and to form a whole pattern, a pattern formation process is continuously performed while the stage is moved. Herein, the pattern formation process can be programmed so that it is continuously performed by recognizing a pattern region deviating from the scanning range of the laser scanner 32, expressing the recognized pattern region as a new processing range, and selecting the end point of the previous range as the start point of the new range.

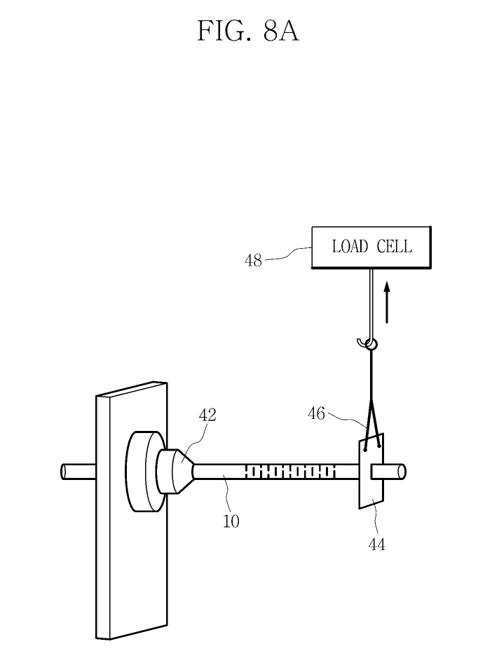

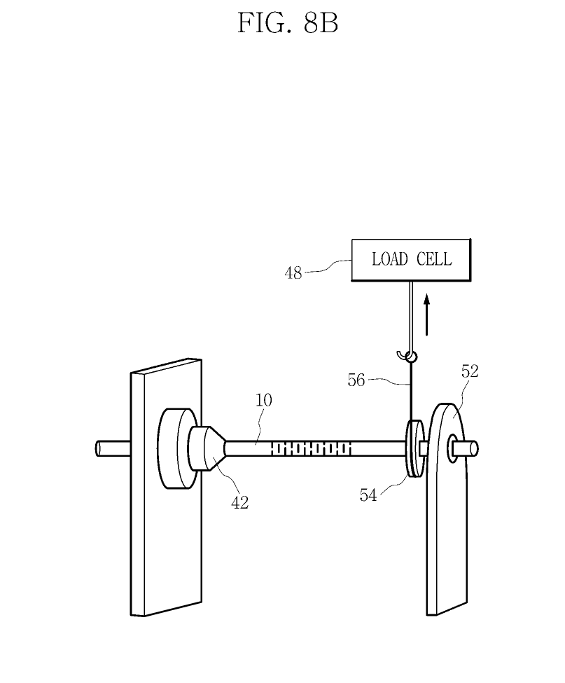

FIGS. 8A and 8B shows test devices for measuring the bending strength and torsional strength of a tube.

Referring to FIG. 8A, the outer circumferential surface of the tube 10 has anisotropic patterns formed thereon. One end of the tube 10 is fixed to a drill chuck 42, and the other end is fitted into a square acryl 44 so that the position thereof is fixed. A connection string 46 is connected to two points of the upper portion of the square acryl 44, and the relationship between force and displacement is measured by a load cell 48 while the connection string 46 is pushed up. When the relationship between force and displacement measured by the load cell 48 is measured, the bending strength of the tube 10 having patterns formed thereon can be determined.

Referring to FIG. 8B, the outer circumferential surface of the tube 10 has anisotropic patterns formed thereon. One end of the tube 10 is fixed to a drill chuck 10, and the other end is supported by a bearing support 52 containing a bearing therein. In addition, a portion of the tube 10, which is near the bearing support 52, is fitted into a circular acryl 54. A connection string 56 is wound around the circular acryl 54, and the relationship between rotating force and displacement by measured with a load cell while the connection string 56 is pushed up. When the relationship between rotating force and displacement measured by the load cell 48 is analyzed, the torsional strength of the tube 10 having patterns formed thereon can be determined.

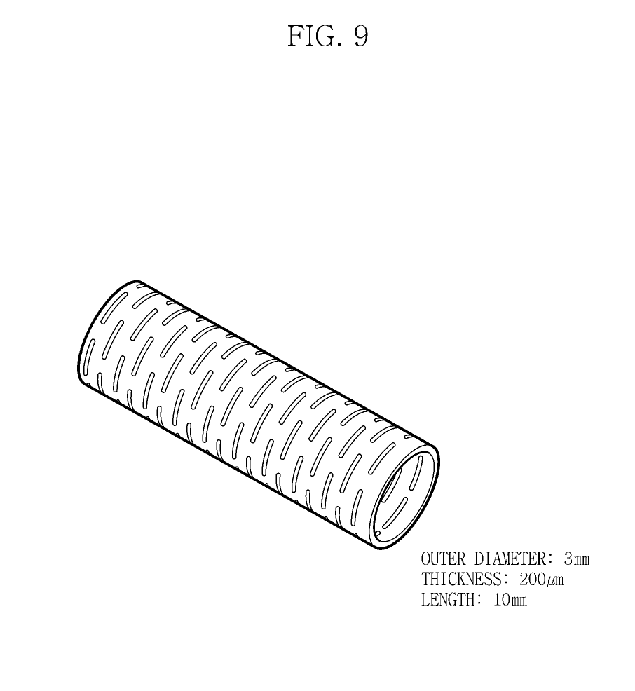

FIG. 9 is a perspective view showing a tube, the bending strength and torsional strength of which are to be measured.

Referring to FIG. 9, a plurality of anisotropic patterns are formed on the outer circumferential surface of the tube along the circumferential direction, and the tube has an outer diameter of 3 mm, a thickness of 200 μm and a length of 10 mm. The bending strength and torsional strength of this tube were compared with those of a tube, which had the same shape as the above tube, but had no pattern.

When the EI value for bending rigidity was compared between the two tubes, EI=Fz³/2d=(0.05 10³)/(2*0.057)=438.6 for the tube having no pattern, and EI=Fz³/2d=(0.05*10³)/(2*0.596)=41.9 for the tube having patterns, suggesting that the EI value for the tube having the patterns decreased by about 90.4%. Herein, d is a displacement value for the force applied in the bending direction.

When the GJ value for torsional rigidity was compared between the two tubes, GJ=ML/θ=Fr²L/d=(2*1.5²*10)/0.149=302 for the tube having no pattern, and GJ=ML/θ=Fr²L/d=(2*1.5²*10)/0.48=93.75 for the tube having the patterns, suggesting that the GJ value for the tube having the patterns decreased by about 68.9%. Herein, d is a displacement value for the force applied in the torsional direction.

When the EI/GJ ratio was compared between the two tubes, EI/GJ=438.6/302=1.45 for the tube having no pattern, and EI/GJ=41.9/93.75=0.45 for the tubes having the patterns, suggesting that the EI/GJ ratio for the tubes having the patterns decreased to about 31.1%. Thus, it was confirmed that the tube having the patterns has advantageous physical property values compared to the tube having no pattern. When the bifurcation problem is solved by forming patterns on the outer circumferential surface of the tube to change physical property values of the tube as described above, the problems of the tube continuum robot, such as high risk and difficult control, can be solved.

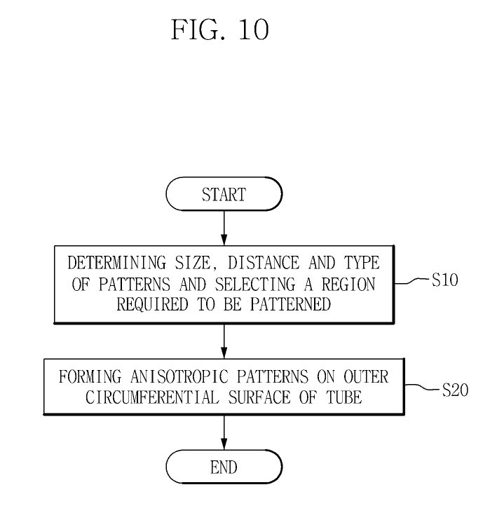

FIG. 10 is a flow chart showing a method for manufacturing a tube having anisotropic patterns according to an embodiment of the present disclosure.

Referring to FIG. 10, the size, distance and type of patterns to be formed on the tube 10 are determined, and a region required to be patterned is selected (S10). These parameters can be determined depending on the physical property values to be imparted to the tube 10.

Then, anisotropic patterns are formed on the outer circumferential surface of the tube 10 (S20). As described above, the anisotropic patterns are formed by a cutting, peeling, etching, deposition or annealing process.

When anisotropic patterns are formed on a tube curved with a predetermined curvature, the sizes, lengths or shapes of the anisotropic patterns formed inside, outside or near the curved portion may differ from each other in order to control the concentration of stress. In addition, before anisotropic patterns are formed on a tube curved with a predetermined curvature, the tube can be straightened by inserting a circular rod therein, followed by patterning.

In addition, when patterning is performed on a straight tube, anisotropic patterns may be formed on the tube by a cutting, peeling, etching, deposition or annealing process as described above, after which the tube may be curved with a predetermined curvature and annealed. The tube manufactured as described above may be used in the manufacture of a tube continuum robot.

Moreover, when the cutting or peeling process is performed using a laser, the cutting or peeling process can be programmed so that it is continuously performed by recognizing a pattern region deviating from the scanning range of the laser scanner, expressing the recognized region as a new processing range, and selecting the end point of the previous range as the start point of the new range.

According to the present disclosure, patterns are formed on the tube 10 so that the tube has desired physical property values. Thus, the present disclosure can be applied in various fields in which tubular mechanical elements are used. For example, the present disclosure can be applied in fields, such as medical robots or fluid transfer systems, in which tubular elements are frequently used.

As described above, the tube continuum robot and the method for manufacturing the tube according to the present disclosure make it possible to manufacture the tube having desired physical property values by suitably controlling the bending strength and torsional strength of the tube. Thus, the bifurcation phenomenon of a tube continuum robot comprising an assembly of these tubes can be alleviated while the robot can be accurately controlled.

Although the preferred embodiments of the present disclosure have been described for illustrative purposes, those skilled in the art will appreciate that various modifications, additions and substitutions are possible, without departing from the scope and spirit of the disclosure as disclosed in the accompanying claims.

What is claimed is:

1. A tube continuum robot having a plurality of overlapping tubes, one or more of the plurality of overlapping tubes having a curved shape, wherein a plurality of anisotropic patterns are formed on an outer circumferential surface of the one or more tubes along a lengthwise or circumferential direction of the tubes.

2. The tube continuum robot of claim 1, wherein the anisotropic patterns are formed by performing a cutting, peeling, etching, deposition or annealing process on the outer circumferential surface of the tubes.

3. The tube continuum robot of claim 1, wherein a ratio of an area or length of each of the anisotropic patterns along the lengthwise direction of the tubes to an area or length of the anisotropic pattern along the circumferential direction of the tubes is controlled so that a ratio at which a bending rigidity and torsional rigidity of the tubes are decreased is controlled.

4. The tube continuum robot of claim 3, wherein each of the anisotropic patterns on the tubes is configured such that the area or length of the anisotropic pattern along the lengthwise direction of the tubes is smaller than the area or length of the anisotropic pattern along the circumferential direction of the tubes so that the bending rigidity of the tubes decreases more than the torsional rigidity of the tubes.

5. The tube continuum robot of claim 3, wherein each of the anisotropic patterns on the tubes is configured such that the area or length of the anisotropic pattern along the lengthwise direction of the tubes is larger than the area or length of the anisotropic pattern along the circumferential direction of the tubes so that the torsional rigidity of the tubes decreases more than the bending rigidity of the tubes.

6. The tube continuum robot of claim 1, wherein the anisotropic patterns are formed to be inclined at a predetermined angle with respect to the lengthwise direction or circumferential direction of the tubes.

7. The tube continuum robot of claim 1, wherein both ends of the anisotropic patterns are bent or have a circular shape.

8. A method for manufacturing a tube which is used in a tube continuum robot having a plurality of overlapping tubes, the method comprising: determining the size, distance and type of patterns to be formed on the tube, and selecting a region required to be patterned; and forming a plurality of anisotropic patterns on the outer circumferential surface of the tube along the lengthwise or circumferential direction of the tube.

9. The method of claim 8, wherein forming the plurality of anisotropic patterns comprises performing a cutting, peeling, etching, deposition or annealing process on the circumferential surface of the tube.

10. The method of claim 9, wherein forming the plurality of anisotropic patterns is performed by the cutting or peeling process using a laser, in which the cutting or peeling process is programmed so that it is continuously performed by recognizing a pattern region deviating from the scanning range of the laser scanner, expressing the recognized region as a new processing range, and selecting an end point of a previous range as a start point of the new range.

11. The method of claim 8, wherein forming the plurality of anisotropic patterns is performed so that a ratio of an area or length of each of the anisotropic patterns along the lengthwise direction of the tubes to an area or length of the anisotropic pattern along the circumferential direction of the tubes is controlled so that a ratio at which a bending rigidity and torsional rigidity of the tubes are decreased is controlled.

12. The method of claim 8, wherein forming the plurality of anisotropic patterns comprises forming anisotropic patterns on a tube curved with a predetermined curvature, in which the sizes, lengths or shapes of the anisotropic patterns inside, outside and near the curved portion differ from each other.

13. The method of claim 8, wherein forming the plurality of anisotropic patterns comprises, before forming patterns on a tube curved with a predetermined curvature, straightening the curved tube by inserting a circular rod therein.

14. The method of claim 8, wherein forming the plurality of anisotropic patterns comprises forming anisotropic patterns on a straight tube by a cutting, peeling, etching, deposition or annealing process, curving the tube having the anisotropic patterns with a predetermined curvature, and annealing the curved tube.
